(19) United States
(12) Patent Application Publication
(10) Pub. No.: US 2004/0219212 A1
(43) Pub. Date: Nov. 4, 2004

(54) Single-daily dose antidiabetic oral pharmaceutical form comprising a biguanide and at least another active principle

(76) Inventors: Catherine Castan, Orlienas (FR); Gerard Soula, Meyzieu (FR); Remi Meyrueix, Lyon (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/478,420

(22) PCT Filed: May 23, 2002
(86) PCT No.: PCT/FR02/01745

(30) Foreign Application Priority Data
May 23, 2001 (FR) 01/06854

(51) Int. Cl. A61K 9/24

(52) U.S. Cl. 424/471

(57) ABSTRACT

An antidiabetic (type II diabetes) oral pharmaceutical form containing an active principle A which is a biguanide such as metformin and at least another active principle B, capable of being easily swallowed, in a single daily dose. The antidiabetic active principle B may be glibenclamide, pioglitazone hydrochloride, rosiglitazone maleate, nateglinide, glipizide or glimepiride. A capsule having a core based on metformin and a coating film applied on the core which enables prolonged release in vivo of metformin is disclosed. The capsule may optionally be used with capsules based on coated active principle B, the coating enabling prolonged release of B. The capsules are designed such that the delivery rate of the galenic form is a single daily dose.

The invention relates to oral galenic forms (tablets, gelatin capsules, powders and the like) in which several antidiabetic active principles are combined and whose daily rate of administration is as small as possible, preferably equal to a single daily intake.

The active principles involved are antidiabetics, and more precisely a biguanide, preferably metformin.

The invention relates to an antidiabetic (type II diabetes) oral galenic form comprising an active principle A chosen from biguanides, metformin being particularly preferred, A being combined with at least one other antihyperglycemic active principle B.

The pathologies which are most particularly of interest in the context of the invention are noninsulin-dependent type II diabetes. In this type II diabetes, hyperglycemia is observed in the patients which finds its origin in a deficiency in insulin secretion by the pancreatic β cells, together with resistance to insulin and reduced glucose tolerance.

Diabetes, and in particular type II diabetes, is a chronic disease which causes serious complications, in particular at the microvascular, neurological and macrovascular level.

Among the microvascular complications, there may be mentioned dysfunction in capillary blood vessels in the retina, the kidneys and the nerves. Retinopathies and nephropathies are well known sequelae of noninsulin-dependent diabetes.

The neurological complications involve the peripheral nervous system (paralysis, pain, sensory deficiency, muscle atrophy and the like) and the autonomous nervous system (diabetic diarrheas, loss of cardiovascular reflexes, bladder and stomach disorders, impotence and the like).

The macrovascular complications are in particular cerebral and peripheral coronary atherosclerosis and coronary cardiac diseases. Diabetes, and in particular type II diabetes, is a very severe chronic pathology which can be lethal.

The treatment of this disease rests on three bases:

a) daily administration of antidiabetic medicaments,

b) controlled diet,

c) physical exercise.

The most convenient and least painful therapeutic treatment for patients is undoubtedly an oral treatment.

The oral antidiabetics (antihyperglycemics) are chosen, without limitation, from the following families:

SULFONYLUREAS;

glibenclamide, nateglinide, glimepiride, glipizide, gliclazide, tolbutamide, tolazamide, gliquidone and chlorpropamide being more specifically selected;

THIAZOLIDINEDIONES;

rosiglitazone maleate, troglitazone (U.S. Pat. No. 4,572,912), zorglitazone, englitazone, darglitazone, the Mitsubishi product MCC-555 (U.S. Pat. No. 5,594,016), the Glaxo-Welcome product GL-262570 and pioglitazone hydrochloride being more specifically selected;

BIGUANIDES; metformin being more particularly selected;

α-GLUCOSIDASE INHIBITORS; acarbose (U.S. Pat. No. 4,904,769) and miglitol (U.S. Pat. No. 4,639,436);

METIGLINIDES;

repaglinide;

INSULINS;

AND COMBINATIONS THEREOF.

The sulfonlyureas and the biguanides are major oral antidiabetics.

The sulfonylureas act by stimulating the secretion of insulin. Their targets are insulin-producing pancreatic β cells.

The biguanides, such as metformin, inhibit glycogenesis and increase the peripheral use of glucose. The biguanides can only be active in the presence of endogenous insulin.

Since the introduction of these various antidiabetic medicaments, doctors prescribe in particular oral treatments of diabetes which combine these various products. That forces patients to take these combinations of medicaments several times per day. Unavoidably, low compliance (compliance with the dosage) is then observed on the part of the patients, who are often elderly persons. Under these conditions, the oral treatments do not have the expected effects and the patients suffer serious complications, which are recalled above and which are specific to diabetes (in particular type II diabetes).

Thus, for these serious chronic diseases such as type II diabetes, it is clear that compliance is a fundamental parameter for the efficacy of the treatment (prevention of serious disorders caused by hyperglycemia and survival of the patient).

By improving compliance, dosage errors and their trail of deleterious effects would be limited. Moreover, the comfort of the patients could only be made better as a result.

The critical importance which a simplified therapeutic and galenic solution, limited to a single daily intake, of a combination of several complementary antidiabetic medicaments, validated by clinical use and clinical studies, is thus measured.

However, to arrive at this ideal solution, it is advisable to solve a good number of technical problems, some of which are indicated below.

The first problem with which researchers are confronted is that of the differences between the daily rates of administration of oral antidiabetics. This constitutes a check on the combinations of various active principles in a single oral galenic form.

Indeed, in the case of a biguanide such as metformin, it is an antidiabetic active principle whose immediate release form has a rate of administration of two daily intakes, whereas other classes of antidiabetics such as sulfonylureas (glibenclamide) are administered orally once per day.

It can therefore be understood that, in this example, metformin and glibenclamide can only be combined in a single oral galenic form provided the duration of action in vivo (bioavailability) of metformin is increased, so as to bring the rate of administration of metformin to a single daily intake, without modifying the behavior of the associated active principle, in this case glibenclamide.

Another prerequisite for the therapeutic and galenic solution intended above involves the use of the combination of two compatible active principles, so as to ensure the stability during storage of the pharmaceutical form considered. The two active principles should not be subject to interruptions leading to degradations.

Another critical galenic point is to minimize the phenomena of release of massive doses of active principles, locally and in a prolonged manner, in the gastrointestinal tract (“dose dumping”). These phenomena are responsible for serious gastrointestinal disorders, such as gastric ulcerations.

The unpleasant taste of certain active principles is such as to disrupt compliance in certain patients. It is therefore important to offer galenic solutions which allow masking of the taste of the active principles.

It is also advisable that the oral pharmaceutical form (tablet, gelatin capsule, powder or sachet) which it is desired to produce is easy to swallow, including for elderly persons.

There are, in this context, a number of previous technical proposals which have vainly sought to solve the problematics described above.

Thus, European patent application EP-A-0 974 356 which describes tablets comprising a combination of metformin and glibenclamide, in which the size of the particles of glibenclamide is such that at most 10% of the particles have a size of less than 2 μm and that at most 10% of these particles have a size greater than 60 μm.

The metformin/glibenclamide tablet is obtained by compressing:

granules based on polyvinylpyrrolidone (66.6 g), metformin (1 500 g), glibenclamide (16.5 g with 10 to 90% of the particles having a size between 2 and 60 μm), croscarmellose sodium (42 g) and microcrystalline cellulose (284.4 g);

microcrystalline cellulose (97.5 g);

magnesium stearate (12 g).

The tablets are then coated with hydroxypropylmethyl-cellulose. The major disadvantage of these tablets consists in their daily rate of administration, which is two intakes per day, because of the metformin; and in spite of the fact that the glibenclamide alone can be ingested once a day.

This tablet is therefore perfectible in relation to improving compliance.

Furthermore, it is to be feared that the unprotected, uncoated active principles (metformin/glibenclamide) contained in this tablet according to EP-A-0 974 356 interact and break down prematurely during storage, before being ingested.

A biphasic system for the controlled release of metformin is also known from application WO-99/47128. This galenic form comprises only one active principle: metformin. The galenic system considered consists of a tablet comprising an outer matrix phase made of hydroxypropylmethylcellulose and microcrystalline cellulose. Included in this outer phase are granules which form the inner phase and which consist of metformin and ethyl cellulose and carboxymethylcellulose.

This galenic system is designed to have a prolonged residence time in the stomach, without disintegrating.

A significant risk of a massive release of metformin over a limited area of the stomach wall (“dose dumping”) thus exists. Now, this phenomenon generates gastric disorders which are completely undesirable for the patient. This disadvantage is all the less tolerable in a permanent treatment for a chronic disease.

This biphasic matrix system is thought to be unsuitable for receiving another antidiabetic active principle combined with metformin. Indeed, it would be achieving the impossible to succeed in controlling the kinetics of release of the additional active principle, in order to harmonize it with the kinetics of release of metformin. Under these conditions, it would be a priori very delicate to obtain a rate of administration of a daily intake, for both active principles.

In summary, the teaching of this document does not fall within the context of a combination of a biguanide (metformin) A and at least one other antihyperglycemic B, in a single oral galenic form, in a single daily intake. In addition, it does not solve most of the components of the problematics described above.

The same applies to the PCT applications WO-A-98/55107 and WO-A-99/47125, which also relate to monolithic galenic systems, having the size of a tablet (≈10 mm), containing only metformin as antidiabetic active principle.

In this state of the art, one of the main objectives of the present invention is to solve the problematics mentioned above, which are to provide an antidiabetic (type II diabetes) oral pharmaceutical form:

containing an active principle A consisting of metformin and at least one other active principle B,

and capable of being easily swallowed, once per day.

Another main objective of the invention is to provide an oral pharmaceutical form based on metformin A combined with another antidiabetic active principle B (promoter of the action of A), allowing simplification and improved compliance, without these gains being made at the expense of therapeutic efficacy.

Another main objective of the invention is to provide a “single daily intake” oral pharmaceutical form based on metformin A combined with at least one other anti-diabetic active principle B which is a promoter of the action of A, without neglecting the undesirable (“dose dumping”) and economic gastric effects, in this search for a galenic solution to the above-mentioned problematics.

Another main objective of the invention is to provide a “single daily intake” oral pharmaceutical form based on metformin A combined with another antihyperglycemic active principle B, using harmless pharmaceutical aids (excipients) which have been approved as such by the regulatory authorities.

Another main objective of the invention is to provide a “single daily intake” oral pharmaceutical form based on metformin A combined with another antihyperglycemic active principle B, which is easy to swallow.

Another main objective of the invention is to provide a polytherapy (bitherapy) for diabetes (in particular type II diabetes) comprising metformin A and at least one other active principle B and provided in the form of a galenic entity administered once per day, in which the deleterious interactions between metformin A and the active principle B are avoided during storage.

Another main objective of the invention is to provide an oral galenic form based on metformin A and at least one other active principle B, in which the taste of A and optionally of B is masked.

These objectives, among others, are achieved by the invention which relates to an oral pharmaceutical form comprising in particular a combination:

of an active principle A consisting of a biguanide, preferably metformin,

and of at least one other active principle B different from A and chosen from antidiabetics, preferably antihyperglycemics, whose rate of administration is one or more intakes per day,

characterized:

[symbol] in that it contains:

a plurality of capsules each consisting of a core based on metformin A and of a film of coating applied to the core and allowing the prolonged release in vivo of metformin A;

and optionally, in the case where the rate of administration of the active principle B is equal to several doses per day, a plurality of capsules each consisting of a core based on an active principle B and a film of coating applied to the core and allowing prolonged release in vivo of the active principle B;

[symbol] and in that the capsules based on A and the optional capsules based on B are designed such that the rate of administration of the galenic form considered is a single daily intake.

In accordance with the invention, an antidiabetic oral galenic form based on metformin A and at least one other antidiabetic active principle B has been advantageously successfully developed in which the daily rate of administration of the metformin, and optionally the daily rate of administration of the active principle B, was (were) adjusted to a single daily intake, by virtue of the use of capsules based on A, or even of capsules based on B, coated and individualized. This coating has a structure and a composition which allow the prolonged release in vivo of the active principles A, or even B, and therefore indirectly the extension of the duration of action of A, or even of B to be regulated.

Thus, if the rate of administration of A is two daily intakes, while that of B is a single daily intake, the metformin A is made in the form of coated capsules and it is combined with the active principle B. The latter is free of a conversion such as to modify its rate of release in vivo and its bioavailability. It is therefore possible to prepare tablets, gelatin capsules or sachets of powder comprising the required daily doses of A and B and whose ingestion once per day is easy, which optimizes compliance.

Moreover, the fact that the metformin A, or even the active principle B, is encapsulated makes it possible to avoid any possible harmful interaction between A and B during storage.

Moreover, the galenic forms according to the invention are not large-sized monolithic galenic forms capable of becoming blocked in the twists and turns of the gastro-intestinal tract, thus with the risk of being responsible for a massive and very localized release of the active principles A and B (“dose dumping”). In this scenario, the active principles A and B are not only not absorbed according to the desired profiles, but are moreover capable of causing serious local lesions.

Another advantage of the antidiabetic galenic form A, B according to the invention is to allow, by virtue of the existing coating, prolonged release, masking of the taste of metformin A, or even of the active principle(s) B.

Finally and moreover, the daily rates of administration of a single daily intake are perfectly consistent with expectations in the area of compliance and therefore of compliance with dosages, which is important in this chronic disease which diabetes, and in particular type II diabetes, is.

According to a first embodiment of the invention, the capsules based on A and the optional capsules based on B are microcapsules. These microcapsules are characterized by a particle size between 50 and 1 000 μm, preferably between 100 and 750 μm, and still more preferably between 200 and 500 μm.

According to a second embodiment of the invention, the capsules based on A and the optional capsules based on B are macrocapsules, which are also called “pellets”. These macrocapsules are characterized by a particle size greater than 1 mm, preferably between 1 mm and 10 mm, and still more preferably between 1 mm and 5 mm.

The coating of the capsules is an important component of the pharmaceutical form according to the present invention since it governs the prolonged kinetics of release in vivo of the active principles A, or even B, contained in the core of the capsules. In fine, the coating determines the duration of action of the active principles A, or even B, and therefore the daily rate of administration of a single daily intake.

The composition of this coating is therefore crucial.

Preferably, this composition of the film for coating the capsules based on metformin A and the optional capsules based on B, is the following:

1)—at least one film-forming polymer (P1), which is insoluble in the fluids of the tract, present in an amount of 50 to 90, preferably 50 to 80% by weight on a dry basis relative to the total mass of the coating composition and consisting of at least one nonwater-soluble derivative of cellulose, namely ethyl cellulose and/or cellulose acetate;

2)—at least one nitrogen-containing polymer (P2) present in an amount of 2 to 25, preferably 5 to 15% by weight on a dry basis relative to the total mass of the coating composition and consisting of at least one polyacrylamide and/or one poly-N-vinylamide and/or one poly-N-vinyllactam, namely polyacrylamide and/or polyvinylpyrrolidone;

3)—at least one plasticizer present in an amount of 2 to 20, preferably 4 to 15% by weight on a dry basis relative to the total mass of the coating composition and consisting of at least one of the following compounds: glycerol esters, phthalates, citrates, sebacates, esters of cetyl alcohol, castor oil, salicylic acid and cutin;

4)—and optionally at least one surfactant and/or lubricant present in an amount of 2 to 20, preferably 4 to 15% by weight on a dry basis relative to the total mass of the coating composition and chosen from anionic surfactants, namely alkali or alkaline-earth metal salts of fatty acids, stearic and/or oleic acid being preferred, and/or from nonionic surfactants, namely polyoxyethylenated sorbitan esters and/or polyoxyethylenated derivatives of castor oil, and/or among lubricants such as calcium, magnesium, aluminum or zinc stearates, or such as sodium stearylfumarate and/or glyceryl behenate; it being possible for said agent to comprise only one or a mixture of the above-mentioned products.

Such a coating makes it possible to independently regulate the kinetics of release in vivo of A and optionally of B. That is possible in the novel galenic form according to the invention because the discrete and individualized capsules of coated A are simply physically juxtaposed with B in the form of capsules or otherwise. The capsules of coated A and the encapsulated active principle(s) B indeed have kinetics of release and absorption in vivo which are specific to them and which are different from each other.

This multi (micro or macro)capsule system has the advantage of offering a masking of the taste of A, or even of B if necessary, as well as any desirable safety in relation to the phenomenon of “dose dumping”.

The medicament according to the invention is particularly suitable for antihyperglycemic active principles which have the characteristic of having an absorption window situated in the upper parts of the gastro-intestinal tract (stomach and beginning of the small intestine), which are highly soluble in water and whose dosage is of the order of 1 g to 2 g per day, which requires the ingestion of a large mass of product per intake.

This medicament in a “multi(micro or macro)capsule” galenic form composed of a plurality of capsules promotes, for statistical reasons, good absorption in the absorption window and removes the risk of localized accumulation of active principle. The result thereof is an optimum absorption of antihyperglycemics in the absorption window, in a quantity and over a duration such that the therapeutic coverage may be ensured over at least 12 h, with all the desirable therapeutic efficacy (control of glycemia). Indeed, the large number of particles (e.g. of the order of 10 000 for the microcapsules and 100 for the macroparticles) allows a reproducible distribution, thus reducing the risks of hyper- and hypoglycemia.

As a coating variant for the capsules, it is possible to envisage that the film for coating the capsules contains one or more products selected from the group comprising:

film-forming macromolecules, preferably chosen from the group comprising: cellulose ethers, cellulose ethers/esters, cellulose esters, cellulose diesters, cellulose triesters, cellulose acylate, cellulose diacylate, cellulose triacylate, cellulose diacetate and triacetate, cellulose acetate propionate, cellulose acetate butyrate, polymethacrylates, waxes, copolymers of vinyl acetate;

ethyl cellulose, Eudragite® RS, Eudragite® RL, cellulose acetate being particularly preferred;

plasticizers, preferably chosen from the following nonexhaustive list: acetyl tributyl citrate, acetyl triethyl citrate, acetylated glycerides, castor oil, dibutyl phthalate, diethyl phthalate, diethyl sebacate, dibutyl sebacate, dimethyl phthalate, glycerol, glyceryl monostearate, glyceryl triacetate, polyethylene glycol, polyoxyethylene/polyoxypropylene copolymers, propylene glycol, tributyl citrate, triethyl citrate, adipate, azelate, enzoate, citrate, citric acid esters, triacetin, vegetable oils, glycerin sorbitol, diethyl oxalate, diethyl malate, diethyl fumarate, dibutyl succinate, diethyl malonate, dioctyl phthalate, glyceryl tributyrate;

and optionally other excipients selected from soluble and insoluble fillers (talc, mineral salts, sugars, polyvinylpyrrolidone, polyethylene glycol and the like), lubricants, colorants or pigments.

Still more preferably, the coating of the capsules of A and of the optional capsules B has the following composition:

1—ethyl cellulose

2—polyvinylpyrrolidone

3—castor oil

4—magnesium stearate.

It should be noted that in the case where the active principle(s) B have a rate of administration of a single daily intake, it (they) is (are) not coated with a coating allowing prolonged and controlled release. It is an active ingredient for immediate release. For reasons relating not to the kinetics of release, but to the galenic formulation, this active principle may nevertheless be coated with a protective coating, with no effect on the immediate release kinetics. Such a neutral coating for example consists of:

[symbol] sugars such as sucrose, glucose, lactose, maltitol, mannitol, isomalt, sorbitol, xylitol, starch hydrolysates,

[symbol] gelatin,

[symbol] alginate,

[symbol] gums such as acacia gum,

[symbol] waxes such as carnauba wax,

[symbol] polyvinyl alcohol,

[symbol] polyethylene glycols,

[symbol] poloxamers,

[symbol] polyvinylpyrrolidone,

[symbol] hydroxypropylmethylcellulose, methyl cellulose, hydroxypropylcellulose, carboxymethylcellulose, methyl cellulose, cellulose acetate/phthalate, hydroxypropylmethylcellulose phthalate, hydroxypropylmethylcellulose acetate succinate,

[symbol] Eudragit E, and the like.

This involves customary galenic practices.

The galenic form according to the invention may also be defined by characteristics of release in vitro of the antihyperglycemic active principle(s). It follows therefrom that in a test of dissolution in vitro called type II dissolutest in accordance with the pharmacopeia, the dissolution of the antihyperglycemic active principle(s) extends over at least 8 hours, preferably at least 20 hours.

To detail somewhat the structure of the capsules, it is specified that the core of said capsules may be for example:

a granule containing the antihyperglycemic active principle and granulation excipients

and/or a particle of antihyperglycemic active principle, preferably a monocrystal.

In the core of the capsules, the antihyperglycemic active principle may be combined with one or more excipients. That is in particular the case when the core consists of a granule. The excipients and the methods of granulation used are those which are traditional in granulation.

The granulation excipients used are well known to persons skilled in the art and are in particular those exemplified above.

In practice, the film coating deposited on each granule may consist of one or more film-forming macromolecules such as those mentioned above.

Persons skilled in the art know several film-coating technologies. By way of example, there may be mentioned that involving a Wurster® system from the company Glatt or a Precisioncoater system from the company Aeromatic.

As regards the active principle A, the term “metformin” denotes metformin and its salts, such as metformin hydrochloride.

As regards the active principle(s) B it (they) is (are) chosen, without limitation, from the group of families of antihyperglycemic agents comprising:

SULFONYLUREAS;

glibenclamide, nateglinide, glimepiride, glipizide, gliclazide, tolbutamide, tolazamide, gliquidone and chlorpropamide being more specifically selected;

THIAZOLIDINEDIONES;

rosiglitazone maleate, troglitazone, zorglitazone, englitazone, darglitazone, the Mitsubishi product MCC-555, the Glaxo-Welcome product GL-262570 and pioglitazone hydrochloride being more specifically selected;

α-GLUCOSIDASE INHIBITORS;

acarbose or miglitol;

METIGLINIDES;

repaglinide;

INSULINS;

AND COMBINATIONS THEREOF.

The galenic system according to the invention may be provided in the form of a galenic unit of suitable mass and volume to allow, on each daily oral administration, the absorption of the required respective daily doses d_{A} and d_{B} of active principles A and B.

In practice, the multimicrocapsule oral pharmaceutical form according to the invention consists of a tablet, a gelatin capsule or powder (packaged in a sachet). These galenic units comprise, as main constituents, dissociated physically and from the point of view of the kinetics of release, capsules of metformin A and particles of B in capsule form or otherwise.

More precisely, the relevant galenic units may be in particular:

tablets capable of disintegrating in the mouth,

effervescent tablets,

tablets capable of disintegrating in a liquid (water),

powders packaged in a sachet of given doses,

suspensions of capsules in a liquid (water),

or gelatin capsules containing a powder of capsules.

To illustrate the invention at the quantitative level, there may be mentioned by way of examples, without limitation, galenic units based on capsules of metformin A, in which:

when B=glibenclamide:

the daily doses required for A and B, d_{A} and d_{B} respectively, are such that:

250 mg≦d_{A}≦2 000 mg 1.25 mg≦d_{B}≦20 mg

for example

d_{A}=1 000 mg and d_{B}=10 mg or 5 mg

when B=pioglitazone hydrochloride:

the daily doses required for A and B, d_{A} and d_{B} respectively, are such that:

250 mg≦d_{A}≦2 000 mg

10 mg≦d_{B}≦45 mg

for example

d_{A}=1 000 mg and d_{B}=30 mg or 15 mg

when B=rosiglitazone maleate:

the daily doses required for A and B, d_{A} and d_{B} respectively, are such that:

250 mg≦d_{A}≦2 000 mg

1 mg≦d_{B}≦20 mg

for example

d_{A}=1 000 mg and d_{B}=8 mg or 4 mg

when B=nateglinide:

the daily doses required for A and B, d_{A} and d_{B} respectively, are such that:

250 mg≦d_{A}≦2 000 mg

100 mg≦d_{B}≦500 mg

for example

d_{A}=1 000 mg and d_{B}=360 mg or 180 mg

when B=glipizide:

the daily doses required for A and B, d_{A} and d_{B} respectively, are such that:

250 mg≦d_{A}≦2 000 mg

2.5 mg≦d_{B}≦40 mg

for example

d_{A}=1 000 mg and d_{B}=15 mg or 5 mg

when B=glimepiride:

the daily doses required for A and B, d_{A} and d_{B} respectively, are such that:

250 mg≦d_{A}≦2 000 mg

1 mg≦d_{B}≦8 mg

for example

d_{A}=1 000 mg and d_{B}=8 mg or 4 mg.

According to yet another of its objects, the present invention relates to a method for treating type II diabetes, in which use is made of the oral pharmaceutical form as defined above (polytherapy, preferably bitherapy: metformin A+active principle B).

The examples which follow will make it possible to better understand the invention and to apprehend all these advantages and all its variant embodiments.

### EXAMPLES

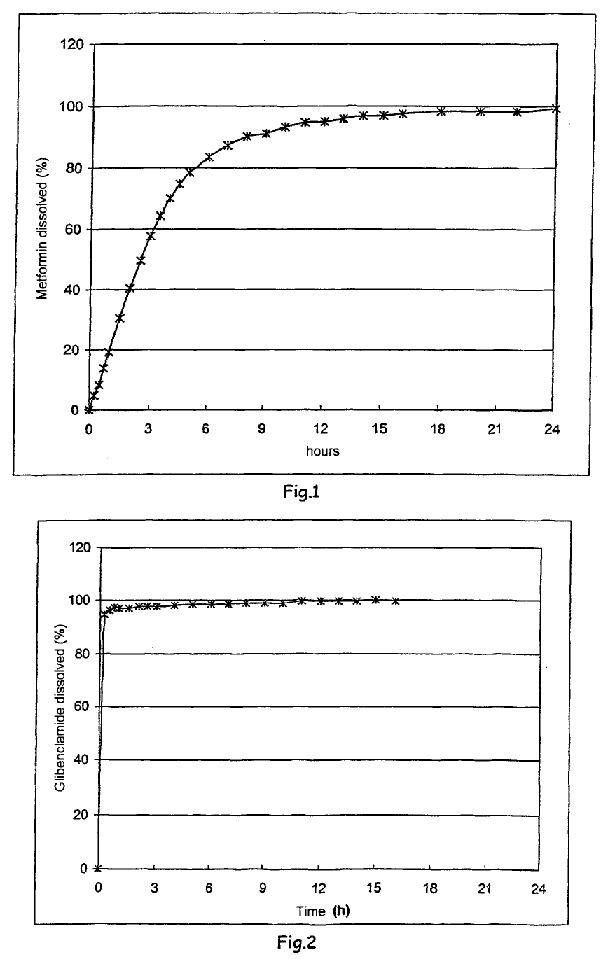

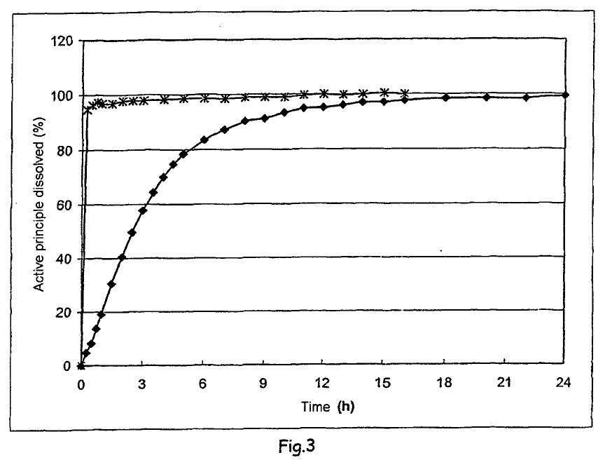

### DESCRIPTION OF THE FIGURES

[symbol] FIG. 1 represents the in vitro dissolution profile of the metformin microcapsules according to example 1, as % of metformin dissolved as a function of time in hours.

[symbol] FIG. 2 represents the in vitro dissolution profile of the glibenclamide microparticles prepared according to example 2, as % of glibenclamide dissolved as a function of time in hours.

[symbol] FIG. 3 represents the in vitro dissolution profile of each of the two active agents (metformin micro-capsules: —♦——♦—)/(glibenclamide microparticles: — [symbol] —— [symbol] —), contained in the gelatin capsules prepared in example 3, as % of active principles dissolved as a function of time in hours.

### Example 1

### Preparation of the Metformin Microcapsules

260 g of ethyl cellulose, 28 g of polyvinylpyrrolidone, 28 of castor oil and 35 g of magnesium stearate are dissolved or dispersed in a mixture consisting of 2 424 g of acetone and 1 616 g of isopropanol. The suspension is sprayed over 1 000 g of metformin/HCl crystals, having a mean diameter between 200 and 500 μm, in a Spray coater Glatt GPCG3. The spray-coating conditions are: product temperature: 38-42° C., spraying rate: 40 g/min, spraying pressure: 3 bar.

The microcapsules obtained were tested in a type II dissolutest in accordance with the pharmacopeia in a KH₂PO₄/NaOH buffer medium at pH 6.8, kept at 37° C. and stirred at 100 revolutions/min.

The dissolution profile obtained is the following:

TABLE 1

| Time | Metformin |
|---|---|
| (hour) | dissolved (%) |
| 2 | 41 |
| 4 | 70 |
| 8 | 90 |
| 12 | 96 |
| 16 | 98 |
| 20 | 99 |

The dissolution profile of the product prepared in this example is represented in the accompanying FIG. 1.

### Example 2

### Preparation of Immediate Release Glibenclamide (Active Principle B) Microparticles

312 g of polyethylene glycol 4000, 78 g of polyvinylpyrrolidone and 43 g of micronized glibenclamide are dissolved or dispersed in 2 450 g of water. The suspension is sprayed over 1 300 g of cellulose microspheres having a mean diameter of between 200 and 500 μm, in a Spray coater Glatt GPCG3. The spray-coating conditions are: product temperature: 38-42° C., spraying rate: 16 g/min, spraying pressure: 5 bar.

The microcapsules obtained were tested in a type II dissolutest in accordance with the pharmacopeia in a KH₂PO₄/NaOH buffer medium at pH 6.8, kept at 37° C. and stirred at 100 revolutions/min.

The dissolution profile obtained is the following:

TABLE 2

| Time | Glibenclamide |
|---|---|
| (hour) | dissolved (%) |
| 0.25 | 95 |
| 0.5 | 96 |
| 1 | 97 |
| 2 | 98 |
| 5 | 99 |
| 12 | 100 |

The dissolution profile of the product prepared in this example is represented in the accompanying FIG. 2.

This profile is characteristic of an immediate release kinetics.

### Example 3

### Preparation of the Final Gelatin Capsule Form

The metformin microcapsules according to example 1 and the glibenclamide microparticles according to example 2 are mixed in the ratio 11.26 to 1. The mixture is placed into gelatin capsules such that each gelatin capsule contains 675.5 mg of microcapsules according to example 1 and 60 mg of microparticles according to example 2, which represents 500 mg of metformin.HCl and 1.5 mg of glibenclamide respectively.

The gelatin capsules obtained were tested in a type II dissolutest in accordance with the pharmacopeia in a KH₂PO₄/NaOH buffer medium at pH 6.8, kept at 37° C. and stirred at 100 revolutions/min.

The dissolution profile obtained for each of the two active agents is in accordance with that obtained from the microcapsules based on metformin and the micro-particles based on glibenclamide, taken separately:

TABLE 3

| | Glibenclamide | Metformin |
|---|---|---|
| Time (hour) | dissolved (%) | dissolved (%) |
| 2 | 98 | 40 |
| 4 | 99 | 71 |
| 8 | 100 | 92 |
| 12 | 100 | 97 |
| 16 | 99 | 100 |
| 20 | 100 | 101 |

The dissolution profile for each of the two active agents contained in the gelatin capsules prepared in this example is represented in the accompanying FIG. 3.

### Example 4

4.1

A pharmacokinetic study was carried out comparing a galenic form consisting of 1 000 mg of multimicro-encapsulated metformin according to example 1 and two tablets of 500 mg of immediate release metformin, marketed under the registered trademark GLUCOPHAGE® (BMS/LIPHA).

The metformin microcapsules of example 1 and the GLUCOPHAGE® tablets are administered in the evening at meal time to 12 healthy volunteers. Blood samples are collected at 0, 0.5, 1, 2, 3, 4, 5, 6, 8, 10, 12, 16, 20, 24 and 36 hours after administration, for analysis of the plasma metformin concentration.

The principal pharmacokinetic parameters obtained are presented below.

TABLE 4

| | Cmax | Tmax | AUC₀₋ₙ |
|---|---|---|---|
| | (ng/ml) | (h) | (ng · ml⁻¹ · h) |
| Metformin | 996 | 7.33 | 9 488 |
| microcapsules | | | |
| example 1 | | | |
| 1 000 mg | | | |
| GLUCOPHAGE ® | 1 229 | 3.75 | 10 087 |
| 500 mg × 2 | | | |

[symbol] Tmax=time corresponding to the maximum plasma metformin concentration (Cmax).

[symbol] AUC₀₋ₙ=area under the plasma concentration profile between the times t=0 and 36 hours (bioavailability).

It is evident from this study:

that the metformin microcapsules of example 1 are a pharmaceutical form whose rate of administration is a single daily intake;

and that GLUCOPHAGE® 500 mg is a pharmaceutical form whose rate of administration is two daily intakes.

In addition, the above study shows, as is confirmed by the book “Physician's Desk Reference”—55th edition 2001—Ed Medical Economics Company—pages 831 to 835, that GLUCOPHAGE® is a tablet form which can be administered twice per day.

4.2: Glibenclamide

The reference book “Physician's Desk Reference”—-54th edition 2000—Ed Medical Economics Company—teaches on page 2457/fig. a), that the galenic form of immediate release tablets of micronized glibenclamide, containing a dose of 3 mg and marketed under the registered trademark GLYNASE PRESTAB®, is characterized by a rate of administration of a single daily intake.

4.3 Metformin/glibenclamide Gelatin Capsules of Example 3

The same release profile in vitro for metformin is found in FIG. 3 as in example 1 (FIG. 1) corresponding to the metformin microcapsules alone.

The metformin microcapsules of example 1 are a pharmaceutical form whose rate of administration is a single daily intake (therapeutic coverage over 24 h).

Consequently, in the metformin/glibenclamide gelatin capsules of example 3, the metformin microcapsules provide a therapeutic coverage over 24 h.

The same release profile in vitro for glibenclamide is found in FIG. 3 as in example 2 (FIG. 2) corresponding to the glibenclamide microparticles alone.

The gelatin capsules of example 3 therefore exhibit appropriate in vitro profiles for a prolonged release form, providing therapeutic coverage over 24 h, for metformin and glibenclamide.

1-7. (Cancelled)

8. An oral pharmaceutical form combining:
an active principle A consisting of a biguanide, preferably metformin,
and at least one other active principle B different from A and chosen from antidiabetics, preferably antihyperglycemics, whose rate of administration is one or more intakes per day, wherein
in that it contains:
a plurality of capsules each consisting of a core based on metformin A and of a film of coating applied to the core and allowing the prolonged release in vivo of metformin A;
and optionally, in the case where the rate of administration of the active principle B is equal to several doses per day, a plurality of capsules each consisting of a core based on an active principle B and a film of coating applied to the core and allowing prolonged release in vivo of the active principle B; and [symbol]
in that the capsules based on A and the optional capsules based on B are designed such that the rate of administration of the galenic form considered is a single daily intake;
wherein the capsules have a particle size between 50 and 1,000 μm, preferably between 100 and 750 μm, and still more preferably between 200 and 500 μm;
and wherein the composition of the film for coating the capsules comprises:
1)—at least one film-forming polymer (P1), which is insoluble in the fluids of the tract, present in an amount of 50 to 90, preferably 50 to 80% by weight on a dry basis relative to the total mass of the coating composition and comprising at least one nonwater-soluble derivative of cellulose, namely ethyl cellulose and/or cellulose acetate;
2)—at least one nitrogen-containing polymer (P2) present in an amount of 2 to 25, preferably 5 to 15% by weight on a dry basis relative to the total mass of the coating composition and consisting of at least one polyacrylamide and/or one poly-N-vinylamide and/or one poly-N-vinyllactam, namely polyacrylamide and/or polyvinylpyrrolidone;
3)—at least one plasticizer present in an amount of 2 to 20, preferably 4 to 15% by weight on a dry basis relative to the total mass of the coating composition and comprising at least one of the following compounds: glycerol esters, phthalates, citrates, sebacates, esters of cetyl alcohol, castor oil, salicylic acid and cutin;
4)—and optionally at least one surfactant and/or lubricant present in an amount of 2 to 20, preferably 4 to 15% by weight on a dry basis relative to the total mass of the coating composition and chosen from anionic surfactants, namely alkali or alkaline-earth metal salts of fatty acids, stearic and/or oleic acid being preferred, and/or from nonionic surfactants, namely polyoxyethylenated sorbitan esters and/or polyoxyethylenated derivatives of castor oil, and/or among lubricants such as calcium, magnesium, aluminum or zinc stearates, or such as sodium stearylfumarate and/or glyceryl behenate; it being possible for said agent to comprise only one or a mixture of the above-mentioned products.

9. The oral pharmaceutical form of claim 8, wherein the film for coating the capsules contains one or more products selected from the groups comprising:
film-forming macromolecules, preferably chosen from the group comprising: cellulose ethers, cellulose ethers/esters, cellulose esters, cellulose diesters, cellulose triesters, cellulose acylate, cellulose diacylate, cellulose triacylate, cellulose diacetate and triacetate, cellulose acetate propionate, cellulose acetate butyrate, polymethacrylates, waxes, copolymers of vinyl acetate; with ethyl cellulose, Eudragit® RS, Eudragit® RL, cellulose acetate being particularly preferred;
plasticizers, preferably chosen from the following nonexhaustive list: acetyl tributyl citrate, acetyl triethyl citrate, acetylated glycerides, castor oil, dibutyl phthalate, diethyl phthalate, diethyl sebacate, dibutyl sebacate, dimethyl phthalate, glycerol, glyceryl monostearate, glyceryl triacetate, polyethylene glycol, polyoxyethylene/polyoxypropylene copolymers, propylene glycol, tributyl citrate, triethyl citrate, adipate, azelate, enzoate, citrate, citric acid esters, triacetin, vegetable oils, glycerin sorbitol, diethyl oxalate, diethyl malate, diethyl fumarate, dibutyl succinate, diethyl malonate, dioctyl phthalate, glyceryl tributyrate;
and optionally other excipients selected from soluble and insoluble fillers (talc, mineral salts, sugars, polyvinylpyrrolidone, polyethylene glycol and the like), lubricants, colorants or pigments.

10. The oral pharmaceutical form of claim 8, wherein the coating of the capsules based on A and of the optional capsules based on B has the following composition:
1—ethyl cellulose
2—polyvinylpyrrolidone
3—castor oil
4—magnesium stearate.

11. The oral pharmaceutical form of claim 8, wherein the active principle(s) B is (are) chosen, without limitation, from the group of families of antihyperglycemic agents comprising:
SULFONYLUREAS; with glibenclamide, nateglinide, glimepiride, glipizide, gliclazide, tolbutamide, tolazamide, gliquidone and chlorpropamide being more specifically selected;
THIAZOLIDINEDIONES; with rosiglitazone maleate, troglitazone (U.S. Pat. No. 4,572,912), zorglitazone, englitazone, darglitazone, the MITSUBISHI product MCC-555, the GLAXO-WELCOME product GL-262570 and pioglitazone hydrochloride being more specifically selected;
α-GLUCOSIDASE INHIBITORS; acarbose or miglitol;
METIGLINIDES; repaglinide;
INSULINS;
AND COMBINATIONS THEREOF.

12. The oral pharmaceutical form of claim 8, wherein said form is provided in the form of a galenic unit of suitable mass and volume to allow, on each daily oral administration, the absorption of the required respective daily doses d_{A} and d_{B} of active principles A and B.

13. The oral pharmaceutical form of claim 8, wherein said form comprises a tablet, a gelatin capsule or powder.

14. The oral pharmaceutical form of claim 8, wherein:
when B comprises glibenclamide, the daily doses required for A and B, d_{A} and d_{B} respectively, are such that 250 mg≦d_{A}≦2000 mg and 1.25 mg≦d_{B}≦20 mg;
when B comprises pioglitazone hydrochloride, the daily doses required for A and B, d_{A} and d_{B} respectively, are such that 250 mg≦d_{A}≦2000 mg and 10 mg≦d_{B}≦45 mg;
when B comprises rosiglitazone maleate, the daily doses required for A and B, d_{A} and d_{B} respectively, are such that 250 mg≦d_{A}≦2000 mg and 1 mg≦d_{B}≦20 mg;
when B comprises nateglinide, the daily doses required for A and B, d_{A} and d_{B} respectively, are such that 250 mg≦d_{A}≦2000 mg and 100 mg≦d_{B}≦500 mg;
when B comprises glipizide, the daily doses required for A and B, d_{A} and d_{B} respectively, are such that 250 mg≦d_{A}≦2000 mg and 2.5 mg≦d_{B}≦40 mg;
when B comprises glimepiride, the daily doses required for A and B, d_{A} and d_{B} respectively, are such that 250 mg≦d_{A}≦2000 mg and 1 mg≦d_{B}≦8 mg.
